(19) United States
(12) Patent Application Publication
(10) Pub. No.: US 2005/0043794 A1
(43) Pub. Date: Feb. 24, 2005

(54) Aspheric intraocular lens

(76) Inventors: Edward Geraghty, Rancho Santa Marguerita, CA (US); M. Laurent Hoffmann, Aliso Viejo, CA (US); Tuan Anh Nguyen, Orange, CA (US)

Correspondence Address: KATHERINE H. MCGUIRE, ESQ.;PATENT COUNSEL BAUSCH & LOMB, INC., ONE BAUSCH & LOMB PLACE, ROCHESTER, NY, 14604, US

(21) Appl. No.: 10/403,985

(22) Filed: Aug. 22, 2003

(51) Int. Cl. A61F 2/16

(52) U.S. Cl. 623/6.23; 623/6.11

(57) ABSTRACT

An intraocular lens for placement into the anterior chamber of a human eye having an optic with a central axis, an anterior surface and a posterior surface. At least one of the surfaces is a rotationally symmetric shaped surface with a center of curvature on the central axis and an 10 aspheric portion formed in a peripheral area of the spherically shaped surface. An attachment mechanism fastens the intraocular lens between the iris and the cornea.

### FIELD OF THE INVENTION

The present invention pertains to intraocular lenses within the anterior chamber, and more particularly, to aspheric intraocular lenses within the anterior chamber.

### DESCRIPTION OF THE PRIOR ART

The prior art discloses numerous materials that are useful in construction of an IOL. The index of refraction can vary greatly depending upon the type of material used to make an IOL. An example of a material that has been used to make an IOL is acrylic, which has commonly been used in the construction of hard lenses. Acrylic has a high index of refraction, therefore, lenses that are made using acrylic are relatively thin. Other materials with a high index to refraction that are useful in making lenses are certain silicone materials. These silicone materials are used in the manufacture of soft lenses.

The ability to fold the IOL is a desirable feature. In order to implant an IOL during surgery, it is desirable to employ a flexible material allowing the IOL to be folded and inserted through a small incision. There are several materials available to make IOLs, including silicone, hydrogel and acrylic. Silicone has been in use for many years and is a soft material. Acrylic appears to have a lower rate of capsule opacification but is a hard material. Hydrogels have emerged as a very suitable material to use for IOLs because they are biologically similar to the tissue of the human eye and the natural lens.

Hydrogel has become a popular material for making IOLs. Hydrogel has favorable properties that make it desirable for constructing IOLs and inserting these IOLs during surgery. Hydrogel is biologically friendly when implanted inside the human eye. A drawback in using hydrogel as an IOL material is that it has a low index of refraction, and accordingly, lenses made from hydrogel need to be thicker than IOL devices made from materials having a higher index of refraction, such as silicone and acrylic.

The use of intraocular lenses (IOL) has become accepted practice with many different types of lenses having been taught by the prior art. Most prior disclosures have taught IOLs that are intended for implant within the posterior chamber of the human eye behind the pupil as a replacement for the natural crystalline lens. Additionally, various prior art disclosures have taught uses for IOLs within the anterior chamber. These prior teachings have taught IOLs that can be used in the anterior chamber for both pseudophakic and phakic patients. The most common construction of these IOLs typically comprises a central optic with two haptics attached to the optic. The two haptics are used to center the optic and properly position the IOL. For those IOLs that are placed in the posterior chamber, a common procedure is to place the IOLs in the capsule which formerly held the eyes natural crystalline lens, this being the common cataract removal procedure. Other posterior chamber IOLs require proper centering behind the pupil, typically using haptics. Anterior chamber IOLs will be positioned in front of the pupil, again typically using haptics. The IOL has an optic portion that functions as a lens having a surface that is rotationally symmetric about a center point of the optic; this surface can be spherical or aspheric. It is well known within the prior art, that the use of optical surfaces that only contain spherically shaped surfaces result in undesirable effects, such as spherical aberrations. The aberrations typically result when light enters through peripheral portions of the optic. Therefore, there is a shortcoming in using optics with only spherically shaped surfaces.

It is also known within the prior art, to apply an aspheric shape in combination with a spherical shape. These IOLs that include the addition of an aspheric shape to portions of the optic, are typically referred to as aspherical lenses. Aspherical lenses have alleviated many of the problems that were associated with spherical lenses. The aspheric lenses in the prior art have been designed to maximize the focus attributes as a design choice in order to reduce the spherical aberrations that result when light is incident from peripheral areas onto the lens, referred to herein as off axis. In concentrating on the correction of spherical aberrations and focusing attributes as the major design parameters, prior art designs for aspherical lenses have not addressed other potential benefits and can be realized by applying different design parameters to aspheric surfaces. Prior art devices exist for the application of an aspheric shape to intraocular lenses. Therefore, these prior art aspheric intraocular lenses, have typically been used to reduce spherical aberrations or for the application of pseudo accommodation.

In view of the foregoing discussion, there remains a need within the art for intraocular lenses which take advantage of the features of using aspherical surfaces and that can be placed in the anterior chamber of human eye.

### SUMMARY OF THE INVENTION

The present invention addresses the shortcomings within the prior art by providing an intraocular lens for placement into the anterior chamber having an optic with a central axis, an anterior surface and a posterior surface. At least one of the surfaces is a rotationally symmetric surface with a center of curvature along the central axis within an aspheric portion formed in a peripheral area of the rotationally symmetric surface. An attachment mechanism fastens the intraocular lens to the cornea near the iris.

It is an object of the invention to provide an intraocular lens to be placed in the anterior chamber of the human eye that can be made as thin as possible.

It is another object of the invention to provide an intraocular lens that is placed in the anterior chamber of human eye and offer expanded clear focal area over the pupil.

It is still another object of the invention to provide an intraocular lens that can rest comfortably in the anterior chamber of human eye and still provide tract focusing.

### BRIEF DESCRIPTION OF THE DRAWINGS

FIG. 1 is a cross-sectional diagram the human eye containing the IOL of the invention as viewed from the top;

FIG. 2 is a sectional view of the lens envisioned by the present invention.

### DETAILED DESCRIPTION OF THE INVENTION

This invention applies the concepts of aspherical constants chosen to minimize the center thickness of the lens. This invention pertains to the thinning of an anterior chamber of an intraocular lens (IOL) by applying a non-spherical surface to the posterior side of the lens. It has been determined that the form of the non-spherical surface does not degrade the optical performance of the lens due to its aspheric shape profile. Optical simulations show that the lens center thickness can be made thinner by as much as 20% through the application of an aspheric surface.

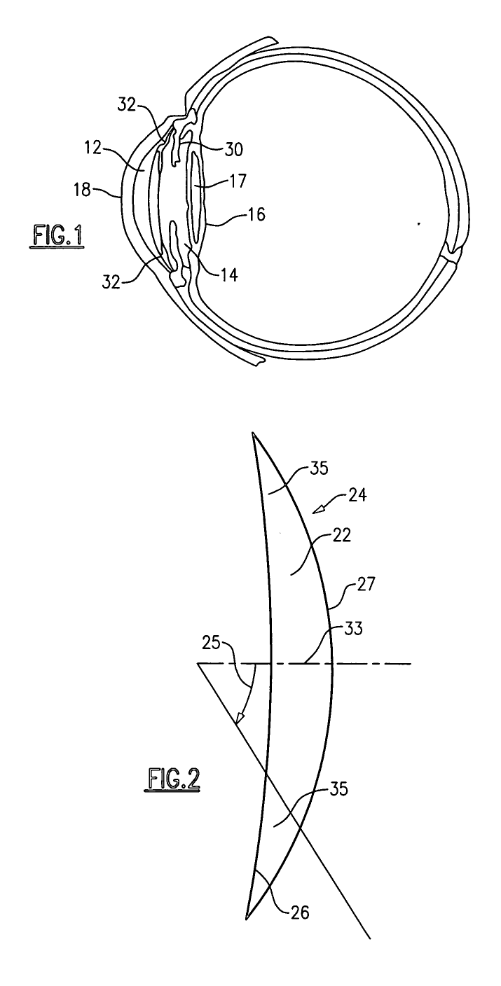

FIG. 1 shows a cross-sectional view of a human eye 10 having an anterior chamber 12 and a posterior chamber 14 separated by the iris 30. Within the posterior chamber 14 is a capsule 16 that holds the eye's natural crystalline lens 17. Light enters the eye by passing through the cornea 18 to the crystalline lens 17 which act together to direct and focus the light upon the retina located at the back of the eye. The most preferred embodiment of the invention is for phakic IOL patients, as illustrated in FIG. 1. However, the invention also specifically envisions that the invention is applicable to pseudophakic patients as well. In the case of pseudophakic patients, the crystalline lens 17 is replaced with an intraocular lens (not shown).

Referring to FIG. 2, the optic 22 of the IOL 24 as envisioned by the present invention has a posterior surface 26 and anterior surface 27. Both the anterior surface 27 and the posterior surface 26 have a radius of curvature 25 relative to the central axis 33 in the central portion of optic 22. An aspheric portion 35 is formed upon optic 22 in the peripheral regions of optic 22. The aspheric portion 35 is designed to be part of optic 22 such that the thickness of optic 22 can be made thin. The thinning of optic 22 can be accomplished while retaining the same focusing characteristics by flattening the base curves to produce a thinner optic 22. It is a well-known fact that there is a difference in sags between spherical and aspherical curves. This difference revolves around the basic fact that for a given diameter an aspheric surface is shallower than a spherical surface.

The aspheric surface sag is of the form:

$$Z = \frac{{cr}^{2}}{1 + \sqrt{1 - ( 1 + k ) {c}^{2} {r}^{2}}} + {α}_{1} {r}^{2} + {α}_{2} {r}^{4}$$

wherein ‘Z’ is the surface sag, ‘k’ is the base conic constant ‘c’ is the surface base radius, ‘r’ is the radial coordinate referenced from the center of the lens and α represents the aspheric coefficients. This is an even form of the aspheric equation.

The invention employs the properties of an asphere to make the aspheric portion 35 operate in conjunction with a central focusing portion along the central axis 33 of optic 22 that allows the resulting optic 22 to be thinner along the central axis 33. There are numerous advantages to making an IOL thinner. A modem surgical procedure for implanting an IOL, involves folding the IOL so that it can be inserted through a small incision; therefore it is desirable to make the lens thinner and easy to fold. Another reason why it is desirable to have thinner IOLs, is that thicker lenses result in discomfort for the patient. For example, hydrogel is one of the most popular types of materials used to make IOLs. A problem in using hydrogel is that hydrogel has a low index of refraction, resulting in the lenses made using hydrogel being thicker lenses than those made with materials having a higher index of refraction. The present invention demonstrates that the aspheric surface sag can be used to create an aspheric surface that flattens the radius of curvature in the peripheral portions of an IOL allowing for a thinner lens diameter in the center without reducing the image quality of the lens. Aspheric lenses have wider ranges and variations in prescriptions that can be applied to them, which allows lenses employing aspheres to take advantage of the above referenced sag equation to provide a great number of potential prescriptions, wherein each of these prescriptions will measure sag for a rotationally symmetric central portion of the IOL compared to the sag of the aspheric portion 35 of the IOL. It will be readily apparent that a vast number of potential prescriptions can be made using these parameters. These prescriptions in accordance with the present invention, will thin the central area of the optic 22 while providing a flattening in the aspheric portion 35. The design trade-off that is made by the present invention is that the aspheric portion 35 is not used to correct off axis aberrations, but instead is used to thin the optic 22 along central axis 33. It has been determined that by applying these design parameters, the lens can be made 20% thinner while still retaining the focal attributes that are provided by aspherical lenses in correcting aberrations.

A further design consideration that is made by the present invention is the angle the aspheric portion 35 makes to the cornea and the iris. These design considerations are implemented by the manner in which the IOL is attached within the anterior chamber. Preferably, to most accurately control the angle aspheric portion 35 makes with the cornea and the iris, is desirable that the point of attachment be where the cornea meets the iris. It is envisioned that attachment mechanisms 32 can control the angle of aspheric portion 35 in relation to both the iris and the cornea. In one preferred embodiment, the attachment mechanisms 32 are inserted into the junction between the iris and cornea. In another preferred embodiment, haptics are used to securely fasten the IOL by attachment of the haptics to the cornea where the cornea meets the iris. It is specifically envisioned by the present invention that specific designs for the IOL will rest the edges of the aspheric portion 35 at the junction of the iris and cornea. In each the foregoing embodiments, it is necessary that the point of attachment take into consideration the angle that the aspheric portion 35 makes with the cornea and with the iris.

The IOL of the invention is intended to be implanted into the anterior chamber 12 by folding the IOL during insertion and passing the folded IOL through a small incision in the cornea. The thinness of the aspherical lens of the invention makes this insertion process easier by having a thinner IOL. It has been discovered that concentrating on the thickness of the lens as the most critical design attribute will still result in improved focus within aspherical lenses. Accordingly, the present invention has concentrated on the thickness of the lens in designing aspherical lenses.

It is envisioned by the present invention that the posterior surface 26 contains the surface defining aspheric portion 35. This is the area of the IOL where the rotational symmetry is steepest. Therefore, the greatest effect is obtained by placing aspheric portion 35 on the posterior surface 26. However, it is also envisioned that placing aspheric portion 35 on anterior surface 27 will also have benefits. Is also envisioned that aspheric portion 35 can be placed in a combination of posterior surface 26 on anterior surface 27. The most preferred embodiment of the invention is for an IOL that functions as an anterior lens is placed in the anterior chamber. However, it is also specifically envisioned by the invention that the thinning attributes of the lens can also be used for placed in the posterior chamber lenses.

An additional benefit to lens design of the present invention is that the clear optical zone that forms a usable portion of the lens increases by the application of the aspheric form to the peripheral portions of the lens. The phakic IOL of the invention gradually flattens the spherical form of the lens toward the periphery portions by the application of the aspheric form, most preferably to the posterior surface but it should also be understood that application of aspheric portion to the anterior surfaces is also envisioned by the invention. The asphere is preferably applied to the posterior surface due to the steeper spherical shape applied to the posterior surface. An asphere on the anterior surface would not produce as great a benefit, however, it is specifically envisioned by the invention that the asphere can be contained on the anterior surface.

1. A method of placing an aspherical intraocular lens into the anterior chamber comprising the steps of:
providing an optic having at least one rotationally symmetric surface near a center of the optic and at least one aspheric surface in a peripheral region to the rotationally symmetric surface; and
placing the optic such that the peripheral region is anchored near a junction between the cornea and the iris.

2. The method of claim 1 wherein the step of providing further comprises forming the aspheric surface on a posterior side of the optic.

3. The method of claim 1 wherein the step of providing further comprises forming the aspheric surface on an anterior side of the optic.

4. The method of claim 1 wherein the step of providing further comprises forming the aspheric surface on both have anterior and a posterior side of the optic.

5. The method of claim 1 wherein the step of providing further comprises forming a clear optical zone that comprises the optic and at least a portion of the aspheric surface.

6. The method of claim 1 wherein the step of providing further comprises forming a plurality of haptics attached to the peripheral region and the step of placing further comprises anchoring the haptics to the cornea near the iris.

7. The method of claim 1 wherein the step of providing further comprises forming attachment mechanisms to a peripheral region which attachment mechanisms can be readily inserted into the junction of the iris and the cornea, and the step of placing further comprises placement of attachment mechanisms into the junction of the iris and cornea.

8. The method of claim 1 wherein the step of providing further comprises creating the aspheric surface with the smaller surface sag than the sag of the rotationally symmetric surface.

9. (cancelled)

10. An intraocular lens for placement into the anterior chamber comprising:
an optic having a central axis, an anterior side and a posterior side; and
a first spherically shaped surface on the anterior side and a second spherically shaped surface on the posterior side, both the first and second spherically shaped surfaces having their curvature centered about the central axis;

11. An intraocular lens for placement into the anterior chamber comprising:
an optic having a central axis, an anterior surface and a posterior surface;
at least one of the surfaces having a spherically shaped surface disposed about the center of curvature on the central axis;
an aspheric portion formed in a peripheral area of the spherically shaped surface; and
an attachment mechanism fastened to the optic.

12. The intraocular lens of claim 11 wherein the spherically shaped surface and the aspheric portion are formed on the posterior surface.

13. The intraocular lens claim 11, wherein both the anterior and the posterior surfaces have central spherically shaped surfaces, and the aspheric portion is formed on the posterior surface.

14. The intraocular lens of claim 11, wherein both the anterior and the posterior surfaces have central spherically shaped surfaces, and the aspheric portion is formed on the anterior surface.

15. The intraocular lens of claim 11, wherein the attachment mechanism places the intraocular lens at a predetermined angle between the cornea and the iris.

16. The intraocular lens of claim 15, wherein the attachment mechanism is formed as part of the aspheric portion.

17. The intraocular lens of claim 15, wherein the attachment mechanism further comprises a plurality of haptics.

18. The intraocular lens of claim 11, wherein the optic is thinned by using the aspheric portion is an extension of a total refractive area of the optic.

19. The intraocular lens of claim 18 wherein the optic is thinned according to the asphere sag equation:
$$Z = \frac{{cr}^{2}}{1 + \sqrt{1 - ( 1 + k ) {c}^{2} {r}^{2}}} + {α}_{1} {r}^{2} + {α}_{2} {r}^{4}$$
wherein ‘Z’ is the sag, ‘k’ is the base conic constant ‘c’ is the surface base radius, ‘r’ is the radial coordinate referenced from the center of the lens and α represents the aspheric coefficients; and
wherein the sag of the spherically shaped surface is greater than the sag of the aspheric surface.
